(19) United States
(12) Patent Application Publication
(10) Pub. No.: US 2021/0022888 A1
(43) Pub. Date: Jan. 28, 2021

(54) Finger Prosthetic

(71) Applicant: New Jersey Institute of Technology, Newark, NJ (US)

(72) Inventors: Ricardo Garcia, Newark, NJ (US); Giovanna Marie Nolan, South Plainfield, NJ (US); Christian Pignataro, Rumson, NJ (US); Madison Taylor, Bloomfield, NJ (US); Ashley Mont, Trenton, NJ (US); Sergei Adamovich, Garwood, NJ (US)

(73) Assignee: New Jersey Institute of Technology, Newark, NJ (US)

(21) Appl. No.: 16/926,939

(22) Filed: Jul. 13, 2020

Related U.S. Application Data
(60) Provisional application No. 62/879,020, filed on Jul. 26, 2019.

(51) Int. Cl. A61F 2/58; A61F 2/80; A61F 2/50; B33Y 80/00

(52) U.S. Cl. CPC A61F 2/586; A61F 2/80; A61F 2002/5073; B33Y 80/00; A61F 2/5046

(57) ABSTRACT

Disclosed is a finger prosthetic is provided that could be attached to an individual's hand. The finger prosthetic includes a midsection, a fingertip portion, and a ring. The midsection, the fingertip portion, and the ring could be 3D printed and customized via 3D scanning The finger prosthetic includes a torsion spring system that comprises a fabricated torsion spring, a cable, and a pin. When the individual flexes their PIP joint, tension is generated in the cable and the cable pulls on the prosthetic fingertip thus flexing the prosthetic DIP joint simultaneously. When the individual wishes to extend the prosthetic DIP joint, he/she simply extends the PIP joint, causing the torsion spring to extend the prosthetic DIP joint. When the PIP joint is at rest, the cable will release the tension and the torsion spring will cause the DIP joint to extend to its upright position.

### CROSS-REFERENCE TO RELATED APPLICATIONS

The present application claims the benefit of the filing date of U.S. Provisional Patent Application No. 62/879,020 filed on Jul. 26, 2019 the disclosure of which is hereby incorporated herein by reference.

### STATEMENT REGARDING FEDERALLY SPONSORED RESEARCH

This invention was made with government support under Agreement No. 90RE5021-04-00 awarded by the National Institute on Disability, Independent Living, and Rehabilitation Research. The government has certain rights in this invention.

### FIELD OF USE

The present disclosure relates to a prosthetic device. In particular, the present disclosure relates to a finger prosthetic designed for partial finger amputees.

### BACKGROUND OF THE INVENTION

Digital amputation is a common injury that affects many individuals worldwide. In the United. States alone, it is estimated that about a quarter of a million individuals have non-thumb digital amputations. These injuries often result in extensive functional disability and a substantial social and economic cost to the society. More importantly, the outcome of digital dysfunction is detrimental to individual's daily activities, such as buttoning a shirt or unlocking a door. Therefore, the overall goal for these individuals is to rebuild a finger with restoration of normal function, stability, length, and sensation.

Amputees often have trouble with performing basic tasks, such as typing on a computer or gripping an item. Several devices exist to assist individuals with amputations. However, there are very few options available for individuals with amputations distal to the proximal interphalangeal (PIP) joint. Additionally, the custom-fit nature of existing prosthetic devices typically requires extensive machining techniques and hands-on labor, which drive up the cost of the prosthetic device.

Most prosthetic devices are produced by creating a mold of the individual's residual limb, which is then used to create a plaster cast. In turn, the plaster cast is then modified as needed before finally pulling a thermoplastic over the plaster cast to create a socket, where the residual limb is inserted. The socket is attached to one or more off-the-shelf and/or custom machined parts.

Often, the socket will not fit comfortably on the first attempt. Consequently, the process is repeated with further alterations made during the plaster cast modification stage. Additionally, the production of the mold often involves discomfort for the individual as it may involve wrapping their residual limb in plaster tape and holding until dry. The plaster cast created is prone to deformation and degradation over time, which may necessitate repeating the process numerous times. The process of iteration and alteration involves time consuming, hands-on skilled labor and thus drives up prosthetic cost.

Furthermore, existing prosthetic devices often use motors and batteries to support the finger through movement. The use of motors and batteries in a finger prosthetic device is undesirable for several reasons. For example, these devices increase complexity, are unreliable and more expensive to maintain (e.g., the motor may eventually fail).

Accordingly, there exists a critical need for a reliable, low-cost prosthetic device that can return normal functionality to an amputee.

### SUMMARY

Shown and described is a finger prosthetic that does not utilize batteries nor motors to power prosthetic movement. The finger prosthetic may be attached to an individual's hand and/or a portion of the amputated finger. Compared to the above prior attempts, the presently disclosed device solves the problems of current state of the art, meets the above requirements, and provides many more benefits.

In one aspect, disclosed is a novel finger prosthetic. In one embodiment, the finger prosthetic includes a midsection, a fingertip portion, and a ring. In this embodiment, the midsection, the fingertip portion, and the ring could be 3D printed.

The finger prosthetic includes a torsion spring system that comprises a fabricated torsion spring, a cable, and a pin. This torsion spring is embedded in the prosthetic distal interphalangeal (DIP) joint and applies a moment sufficient to passively extend said joint. This passive extension is in opposition to the active flexion of the prosthetic DIP joint. The active flexion is body powered; when the user flexes their proximal interphalangeal (PIP) joint, a cable transmits tension through the device which applies a moment in opposition to the torsion spring thus flexing the prosthetic DIP joint. The utilization of the torsion spring system embedded in the hinge is novel in the field of Distal Interphalangeal prosthetics. No known device utilizes such a spring system for the purposes and functions disclosed herein.

In one embodiment, an individual could wear the finger prosthetic by inserting their residual limb into the finger prosthetic. Once inserted, the end of the individual's residual limb is positioned in a socket formed in the finger prosthetic and the ring is positioned around the base of the residual limb. The ring provides stability to the rest of the finger prosthetic by resisting axial and angular displacement. The ring is attached to the midsection and the fingertip portion of the finger prosthetic via two lateral struts in one embodiment. Each strut has a hinge joint aligned with the individual's PIP joint so as not to obstruct flexion of the joint.

The midsection and the fingertip portion could interface via a hinge joint serving as a prosthetic distal interphalangeal (DIP) joint. This hinge joint is passively extended by a 90-degree torsion spring and can be actively flexed via a cable, which transmits tension whenever the individual flexes their PIP joint.

When the individual flexes their PIP joint, tension is generated in the cable and the cable pulls on the prosthetic fingertip thus flexing the prosthetic DIP joint simultaneously. When the individual wishes to extend the prosthetic DIP joint, he/she simply extends the PIP joint, causing the torsion spring to extend the prosthetic DIP joint. When the PIP joint is at rest, the cable will release the tension and the torsion spring will cause the DIP joint to extend to its upright position.

Again, depending on the embodiment, the device uses flexion of the PIP joint at the interface with the midsection to create tension in the cable. This tension compresses the legs of the torsion spring together and forces the device to bend at the positioned joints. When the individuals's appendage is relaxed, the tension in the cable is released, causing the embedded torsion spring to return to its natural position at 90 degrees.

The above objects and advantages are met by the present invention. Any combination and/or permutation of the embodiments is envisioned.

In addition, the above and yet other objects and advantages of the present invention will become apparent from the hereinafter-set forth Brief Description of the Drawings, Detailed Description of the Invention and claims appended herewith. It is to be understood, however, that the drawings are designed as an illustration only and not as a definition of the limits of the present disclosure. These features and other features are described and shown in the following drawings and detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

So that those having ordinary skill in the art will have a better understanding of how to make and use the disclosed composition and methods, reference is made to the accompanying figures wherein:

FIG. 1A shows a sideview and a cross-sectional view of a finger prosthetic, in accordance with one embodiment of the present disclosure; and

FIG. 1B shows perspective views of the finger prosthetic;

FIG. 2A illustrates views of a fingertip portion of the finger prosthetic, the far right view showing interior holes for a cable and a torsion spring housing;

FIG. 2B shows additional views of the fingertip portion;

FIG. 3A shows views of a midsection of the finger prosthetic, the far right showing interior holes for the cable and torsion spring housing, as well as a rounded interface where the residual limb will reside while inside the finger prosthetic;

FIG. 3B shows additional views of the midsection;

FIG. 4A shows views of a ring of the finger prosthetic, the far right showing interior holes for the cable housing;

FIG. 4B shows additional views of the ring;

FIG. 5A shows an exploded view of the finger prosthetic, including the 3D printed fingertip, the midsection, the ring, the fabricated torsion spring, the cable, and the stainless-steel pin;

FIG. 5B shows an exploded view of the finger prosthetic with a nitrile fingertip cover;

FIG. 6A shows the assembly of the midsection and the ring, thereby creating a proximal interphalangeal joint and acting as a hinge where the finger prosthetic will flex with the residual limb;

FIG. 6B is a perspective view of the assembled midsection and the ring, with a wall built into the midsection preventing hyperextension failure at the joint, thus increasing the force generated by the remaining appendage when fully extended, where a similar feature is seen at the hinge of the fingertip portion;

FIG. 7 is a perspective view of the finger prosthetic, the fingertip portion being attached by first inserting the spring into the midsection and the fingertip portion, then sliding the aluminum pin through the holes in the spring, the fingertip portion, and the midsection, where this creates the distal interphalangeal joint of the finger prosthetic;

FIG. 8 is a perspective view of the finger prosthetic, the cable being run from the fingertip, down through the midsection, and the ring, where once the cable is run from top to bottom, the cable is looped through the ring, the midsection, and the fingertip portion, a knot or securing mechanism is tied or disposed between the two ends at the top of the fingertip portion, and where the remaining cable is trimmed; and,

FIG. 9 shows profile views of the finger prosthetic, the left side showing the finger prosthetic with no tension in the cable and a relaxed torsion spring, thus mimicking the relaxation state of the prosthetic, where this state of extension in the finger prosthetic serves to imitate that of a human finger, and where the right side shows the finger prosthetic with tension in the cable and compression in the torsion spring as a result of the cable tension from the residual limb bending within the prosthetic, and where this state of flexion in the prosthetic serves to imitate that of a human finger.

### DETAILED DESCRIPTION

The present disclosure is directed to a new finger prosthetic. Although discussed herein with respect to a finger prosthetic for individuals with amputations distal to the proximal interphalangeal (PIP) joint, it should be understood that the mechanism by which the present invention functions (a torsion spring acted on by a cable in tension with a surrounding hinge system) can be used at other finger joints.

As discussed above, partial hand amputations are the most common amputation, accounting for 75 percent of all traumatic amputations. In 2005, 1.6 million persons were living with the loss of a limb. Over 500,000 people were affected by amputation of the hand or fingers in the United States in 2005. There are very few options available for individuals with amputations distal to the PIP joint. Therefore, the disclosed prosthetic design will serve to return normal functionality to an underserved portion of the finger amputee demographic. There is a limited number of companies producing prosthetics for amputations distal to the PIP joint. These current devices have many limitations as discussed herein that the present device overcomes. Additionally, the custom-fit nature of prosthetics typically requires extensive machining techniques and hands-on labor which drives up the cost of the prosthetic.

In fact, as many as 20% of nonmilitary amputees report an unmet need for rehabilitation services, largely because of inability to pay. In the present design to reduce cost and production time, all major components will be 3D printable. This allows for a more affordable and efficient process for size adjustment, refitting, and production.

In one embodiment, the present finger prosthetic is attached to the outside of the individual's hand. The subject wears the prosthetic by inserting their residual limb into the mechanism. Once inside, the end of the subject's residual limb will be in the device's socket and the base of their residual limb will have a ring around it. The ring provides stability to the rest of the prosthetic by resisting axial and angular displacement. The ring is attached to the body of the prosthetic via two lateral struts. Each strut has a hinge joint aligned with the subject's PIP joint so as not to obstruct flexion of said joint.

As further described herein, the body of the prosthetic device contains two segments: the midsection and the fingertip. These two segments interface via a hinge joint serving as a prosthetic DIP joint. This hinge joint is passively extended by a 90-degree torsion spring and can be actively flexed via a cable which transmits tension whenever the subject flexes their PIP joint. In short, when the subject flexes their PIP joint, tension is generated in the cable and the cable pulls on the prosthetic fingertip thus flexing the prosthetic DIP joint simultaneously. When the subject wants to extend the prosthetic DIP joint, they simply extend their PIP joint, causing the torsion spring to extend the prosthetic DIP joint. When the PIP joint is at rest, the cable will release the tension and the torsion spring will cause the DIP joint to extend to its upright position.

The present device is designed in such a way that production involves significantly less hands-on labor and is less physical invasive for the individual than compared to traditional methods. To produce the prosthetic device, 3D scans of the individual's residual limb are acquired and imported into Computer Aided Design (CAD) software along with the device's assembly.

Once in this CAD environment, the device's size and shape can be easily manipulated to fit the shape of the individual's residual limb. This 3D model can then be 3D printed. The only components which need to be added by hand are the tensile cable, the aluminum pin, and the spring which extends the prosthetic DIP joint. This production process serves to reduce hands-on labor and therefore cost while also reducing invasiveness and improving turnaround times.

The ring, midsection, and fingertip of the prosthetic, depending on the implementation, is constructed from 3D printed pieces and will also include a cable, spring, aluminum pin, and socket portion. Once assembled, the prosthetic can flex and relax by utilizing a cable.

The aforementioned mechanism containing the torsion spring and cable apparatus is a novel feature in the present prosthetic. Other companies have utilized the body powered feature, but competitor's prosthetics rely upon more complicated mechanisms with many moving parts. These mechanisms have more areas of friction, more parts to fabricate, and more failure modes. The present mechanism stresses simplicity to maximize strength and production efficiency while minimizing failure modes. Also, due to the mechanism of the device, the prosthetic can be synthesized without the use of a mold kit, which is typically necessary for other prosthetic syntheses. The dimensions of the prosthetic can be tailored per individual by 3D scanning the hand of the subject and then creating the device in a CAD program. This process saves on both time and money and creates a more accurate blueprint to create the prosthetic with. The cost of fabricating mold kits not only makes it more difficult for the consumer but increases the difficulty of the designer. Having a model of the residual limb in the virtual space allows for real-time fitting and accommodation of the irregular geometries present at the residual limb.

Again, the combination cable and spring system used for flexion and extension of the prosthetic DIP joint is wholly unique from other finger prosthetics. All major components of the device (ring, midsection, fingertip) are designed to be 3D printable. This is notable because if one were to completely 3D printable any other currently commercial finger prosthetic, it would not work as intended because of the complexity of the current prosthetic's working mechanisms and motors. Therefore, the present device is uniquely capable of taking advantage of 3D printing during its production. Most prosthetics are produced by creating a mold of the subjects residual limb which is then used to create a plaster cast which is then modified as needed before finally pulling a thermoplastic over the plaster cast to create the socket (where the residual limb is inserted). This socket is then attached to one or more off-the-shelf and/or custom machined parts.

As previously discussed above, the socket often will not fit comfortably on the first attempt and the process starts again with further alterations made during the plaster cast modification stage. Additionally, the production of the mold often involves discomfort for the subject as it may involve wrapping their residual limb in plaster tape and holding until dry. Problematically, the plaster casts created are prone to deformation and degradation over time which may necessitate starting the process from the beginning. The present device is designed in such a way that producing it involves significantly less hands-on labor and less invasive for the subject than traditional methods.

To produce the device, 3D scans of the subject's residual limb are acquired and imported into Computer Aided Design (CAD) software along with the present device assembly. Once in this CAD environment, the device's size and shape can be easily manipulated to fit the shape of the subject's residual limb. This 3D model can then be 3D printed. Again, the only components which need to be added by hand are the tensile cable and the spring which extends the prosthetic DIP joint. This production process serves to reduce hands-on labor and therefore cost while also reducing invasiveness and improving turnaround times.

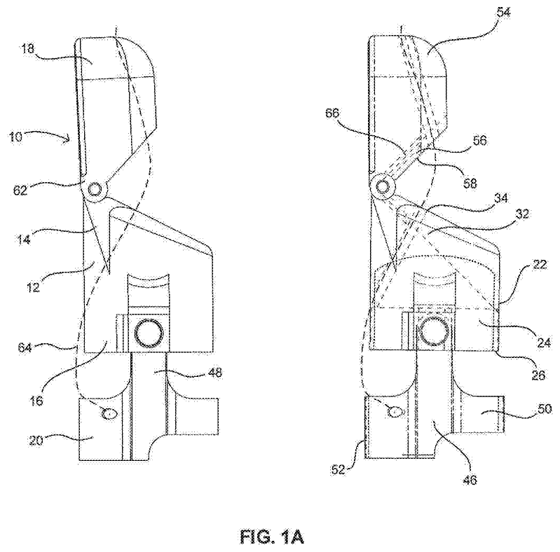

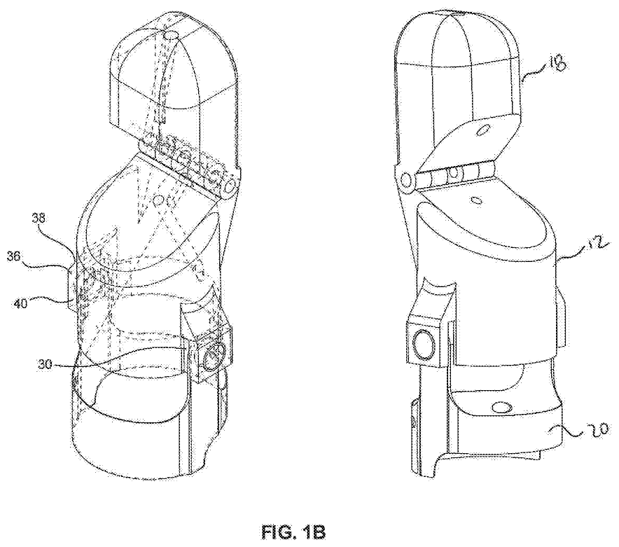

Adverting to the Figures, FIG. 1A shows one embodiment of a finger prosthetic 10. In this embodiment, the finger prosthetic 10 includes a midsection 12 with a top end 14 and a bottom end 16, a fingertip portion 18 connected to the top end 14 of the midsection 12, and a ring 20 removably connected to the bottom end 16 of the midsection 12. As will be described in further detail below, the ring 20 is attached to the midsection 12 by a pair of revolute joints, which are align with the placement of an individual's PIP joint. This allows for the individual to flex their PIP joint unimpeded. The midsection 12 is attached to the fingertip portion 18 by a revolute joint, which simulates the distal interphalangeal (DIP) joint.

The midsection 12 includes a sidewall 22 that defines a chamber 24. The bottom end 16 of the midsection 12 includes an edge 26 that defines an open end of the chamber 24. The sidewall 22 includes a lower substantially cylindrically portion 28 with two diametrically opposed cut-outs 30 (FIG. 1B) and an upper portion 32 with a front downwardly angled wall 34.

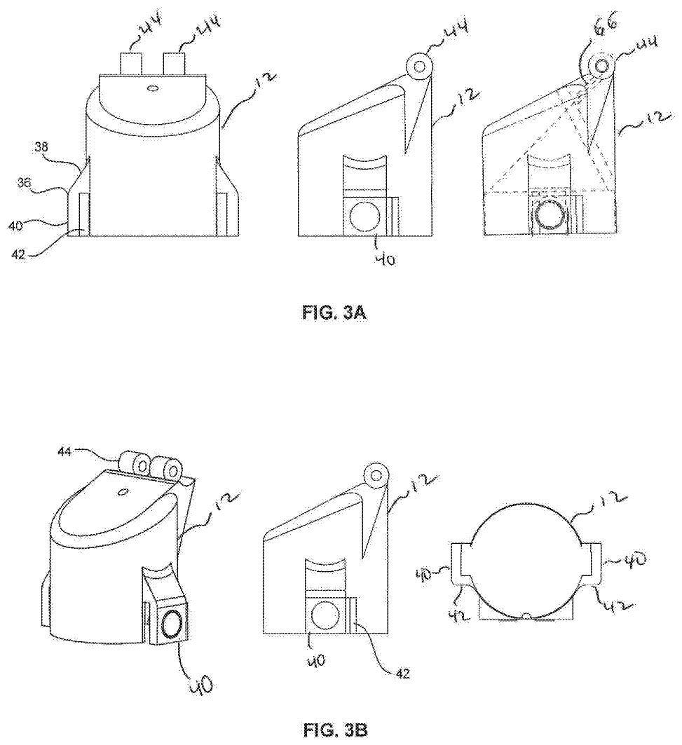

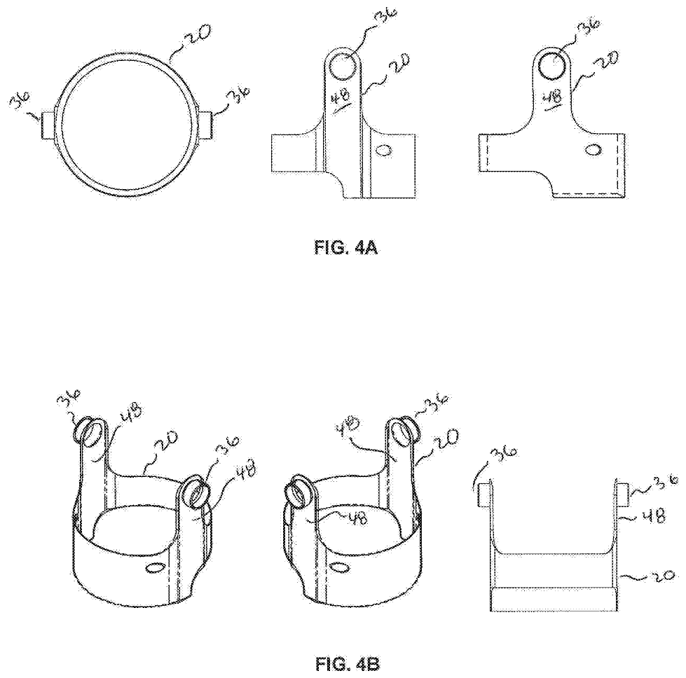

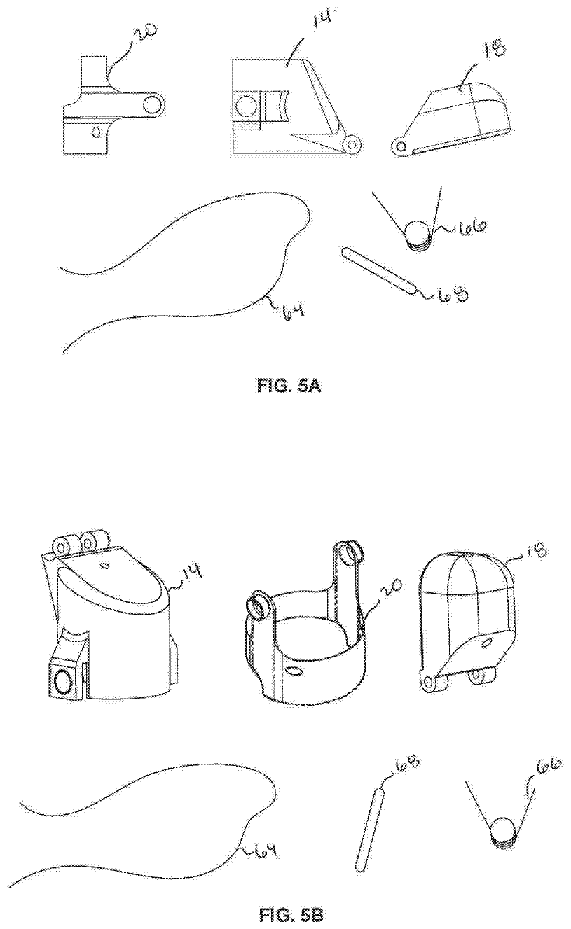

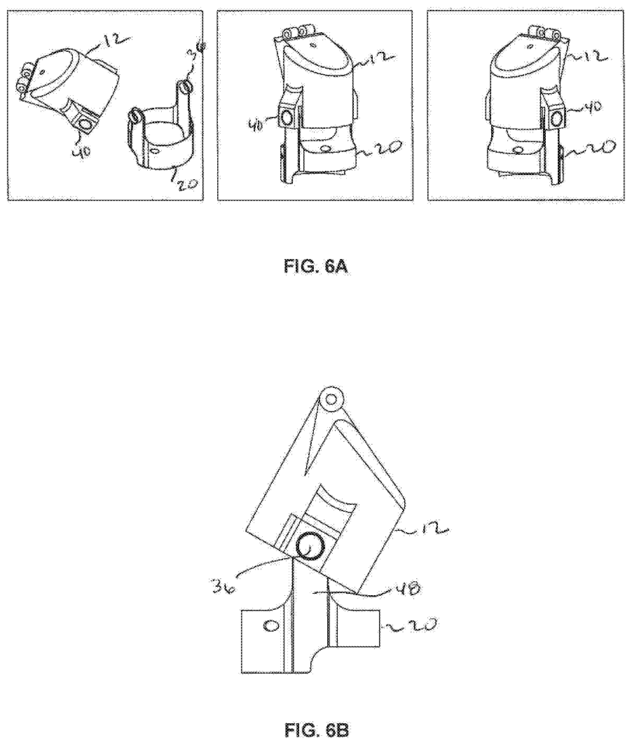

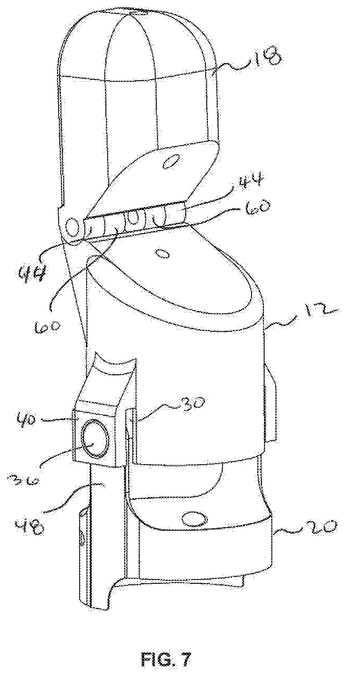

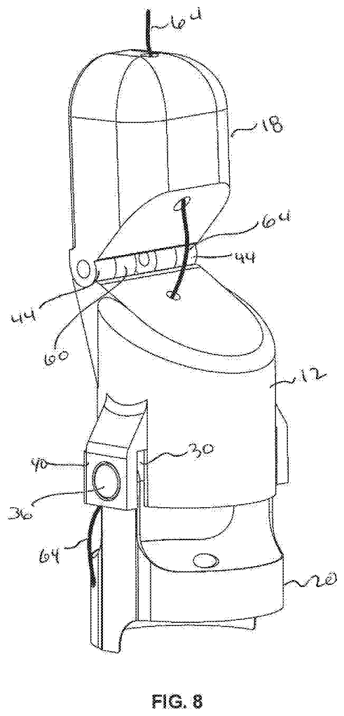

Referring to FIG. 1B, a pair of protrusions 36 extends downwardly from opposite sides of the sidewall 22. Each protrusion 36 includes an angled portion 38 that extend radially outward from the sidewall 22 and an arm 40 that extends vertically from the angled portion 38. Each arm 40 is attached to the sidewall 22 by a ledge 42 (FIG. 3B). Each protrusion 36 is designed for engagement, such as a snap-fit engagement, with the ring 20 such that once engaged, the midsection 12 and the fingertip portion 18 may pivot with respect to the ring 20. In particular, the midsection 12 and the fingertip portion 18 are configured to move between a first position, wherein the midsection 12 and the fingertip portion 18 are in a substantially vertical position, and a second position, where the midsection 12 and the fingertip portion 18 are in a substantially horizontal position. The ledges 42 serve to limit the midsection 12 from pivoting in a rear direction when the midsection 12 is in the substantially vertical position, thereby preventing over-rotation of the midsection 12.

With reference to FIG. 3B, a pair of outer knuckles 44 with apertures is located on the top end of the midsection 12. The outer knuckles 44 cooperate with the fingertip portion 18 to form a revolute joint as will be described below.

Referring to FIG. 1A, the ring 20 includes a sidewall 46 and two diametrically opposed shoulders 48 that extend upwardly from the sidewall 46. The shoulders 48 are designed to interlock with the protrusions 36 of the midsection 12. Although a snap-fit engagement is shown, it will be understood that the midsection 12 and the ring 20 could be attached to each other using any suitable engagement mechanism. In one embodiment, a front section 50 of the ring 20 has a smaller height than a rear section 52 of the ring 20. This configuration allows the midsection 12 to pivot forward between the shoulders until the midsection 12 is in the substantially horizontal position.

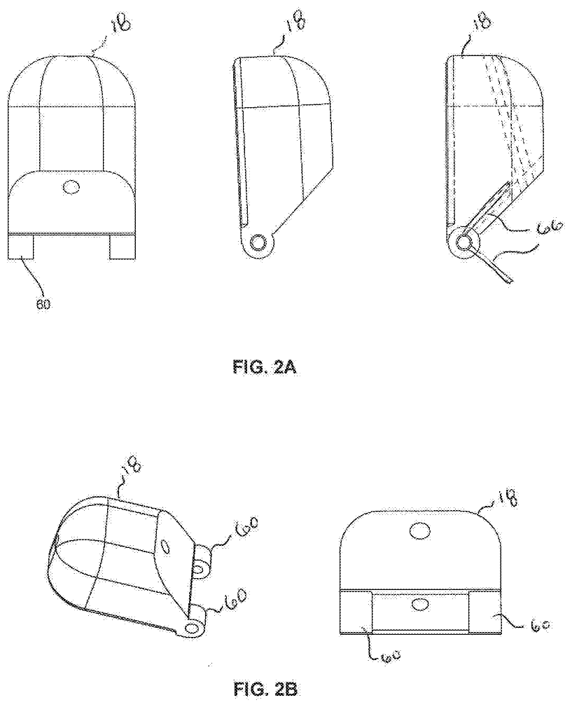

The fingertip portion 18 includes a top end 54 and a bottom end 56 with an upwardly angled wall 58. The bottom end 56 includes a pair of inner knuckles 60 (FIG. 2A) that cooperate with the outer knuckles 44 (FIG. 3B) of the midsection 12 to allow the fingertip portion 18 to pivot with respect to the midsection 12. In particular, the fingertip portion 18 is configured to move between a first position, wherein the fingertip portion 18 is in a substantially vertical position, and a second position, where the angled wall 58 of the fingertip portion 18 moves toward the angled wall 34 of the midsection 12. A pair of ledges 62 serve to limit the fingertip portion 18 from pivoting in a rear direction when the fingertip portion 18 is in the substantially vertical position, thereby preventing over-rotation of the fingertip portion 18.

The finger prosthetic 10 could include a cable 64 made of any suitable material, such as polyethylene, and a torsion spring 66. The cable 64 is attached to the ring 20 and runs through the midsection 12 to its other attachment point at the distal end of the fingertip portion 18. The torsion spring 66 is embedded at ninety degrees in the DIP joint between the midsection 12 and the fingertip portion 18. It will be understood that the torsion spring 66 could be embedded at other angles.

One embodiment of a method to produce the finger prosthetic 10 is discussed below. A 3D scan of the individual's limb is used to acquire the inner dimensions where the residual limb interfaces with the prosthetic. It will be understood that residual limb is defined as the remaining appendage after the injury or amputation occurs. The 3D scan is placed in a computer aided design (CAD) software and used to model the socket of the midsection 12 where the residual limb will be inserted. This method ensures that the shape of the midsection interface and the diameter of the ring 20 provide an appropriate fit.

To maintain average finger length, the dimensions of the finger prosthetic 10 are approximated using the remaining fingers. For example, if an individual is missing their left index finger, the finger prosthetic 10 will approximate the length of the right index finger, if applicable. The midsection 12 and the fingertip portion 18 of the finger prosthetic 10 are modeled to have a length and thickness comparable to those of the individual's intact finger, per previously acquired 3D scans. The ring 20, the midsection 12, and the fingertip portion 18 are 3D printed on a suitable printer, such as a Markforged Mark II printer, using suitable material, such as a Markforged Onyx material, with continuous carbon fiber. In another embodiment, the 3D scan may be performed on a mold that the individual creates at home.

The method allows for at-home substitution of individual parts in the event that a certain part becomes damaged. The fingertip portion 18, the midsection 12, and the ring 20 are 3D printed in one embodiment, which facilitates the printing of a replacement part.

One embodiment to assemble the finger prosthetic 10 is discussed below. The midsection 12 and the ring 20 are snapped into the interlocking mechanisms of the respective parts. This will create the proximal interphalangeal joint and act as the hinge, where the finger prosthetic 10 will flex with the residual limb. The fingertip portion 18 is then attached by first inserting the spring 66 into the midsection 12 and the fingertip portion 18, then sliding the aluminum pin or pin 68 through the holes in the spring 66, the fingertip portion 18, and the midsection 12. This will create the distal interphalangeal joint of the finger prosthetic 10.

The cable is run from the fingertip portion 18, down through the midsection 12, and the ring 20. Once the cable 64 is run from top to bottom, the cable 64 is looped through the ring 20, the midsection 12, and the fingertip portion 18, where a knot can be tied between the two ends at the top of the fingertip portion 18. The remaining cable is trimmed.

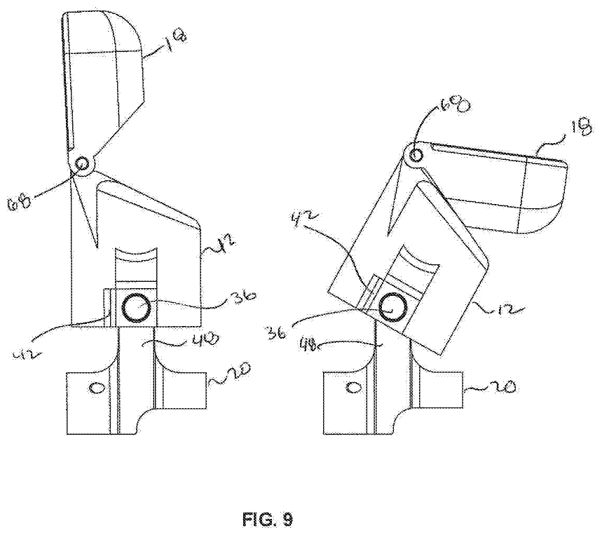

The finger prosthetic 10 uses flexion, as shown in FIG. 9, of the individual's PIP joint at the interface with the midsection 12 to create tension in an ultra-high molecular weight polyurethane cable. This tension compresses the legs of the 90 Degree, 0.105″ OD left-hand torsion spring and causes flexion at the prosthetic DIP joint. When the appendage is relaxed, the tension in the cable is released. This allows the embedded torsion spring to return to its natural position at 90 degrees, thus extending the DIP joint, as shown in FIG. 9.

It will be understood that the present invention will function and can be adapted for any joint in the body, which can be approximated as a revolute joint so long as there is a neighboring proximal joint, which can be flexed to generate tension in the cable. Additionally, the present invention can work for multi joint systems. For example, if an individual undergoes a finger amputation proximal to the PIP joint, the present invention can be used to simulate the motions of both the DIP and the PIP joint. In this case, the tension in the cable to simulate flexion may be generated by the flexion of the wrist or by the flexion of the PIP joint on a neighboring finger.

Although the invention herein has been described with reference to embodiments, it is to be understood that these embodiments are merely illustrative of the principles and applications of the present invention.

It is therefore to be understood that numerous modifications may be made to the illustrative embodiments and that other arrangements may be devised without departing from the spirit and scope of the present invention as defined by the appended claims.

What is claimed is:

1. A finger prosthetic comprising:
a fingertip portion and a midsection flexibly connected together through a hinge joint;
a ring flexibly connected to the midsection by at least one lateral strut;
a torsion spring embedded in the fingertip portion and midsection, the torsion spring configured to allow the fingertip portion to pivot relative to the midsection; and
a cable interwoven through the fingertip portion, the midsection and the ring; wherein when a user flexes a user's proximal interphalangeal (PIP) joint then tension is generated in the cable and the cable pulls on the fingertip portion flexing the midsection and the fingertip portion simultaneously to function as a prosthetic distal interphalangeal (DIP) joint.

2. The finger prosthetic of claim 1, wherein the finger portion, midsection, and ring are all 3D printed and customized via 3D scanning without the use of a plaster cast.

3. The finger prosthetic of claim 1, wherein the ring is positioned is positioned around the base of a residual limb of the user and the ring provides stability to the rest of the finger prosthetic by resisting axial and angular displacement.

4. The finger prosthetic of claim 1, further includes a protrusion disposed on the lateral strut of the ring for creating a hinge connecting the ring to the midsection.

5. The finger prosthetic of claim 4, wherein the protrusion aligned with the user's PIP joint for non-obstruction of flexion of the PIP joint.

6. The finger prosthetic of claim 4, wherein the midsection further includes an arm, the arm defining a hole for connection with the protrusion.

7. The finger prosthetic of claim 6, wherein the arm further includes a cut out portion to provide movement clearance with the lateral strut of the ring.

8. The finger prosthetic of claim 6, wherein the arm is disposed on an outer portion relative to the lateral strut of the ring.

9. The finger prosthetic of claim 1, wherein the hinge joint further includes a pin disposed through the finger portion and the midsection.

10. The finger prosthetic of claim 1, wherein the hinge joint further includes a plurality of inner knuckles disposed on the finger portion and a plurality of outer knuckles disposed on the midsection.

11. The finger prosthetic of claim 1, further includes a pin disposed through the inner knuckles and the outer knuckles.

12. The finger prosthetic of claim 1, wherein the cable is anchored with the finger portion.

13. The finger prosthetic of claim 1, wherein the cable is anchored with the ring.

14. The finger prosthetic of claim 1, wherein the cable is equally anchored with the finger portion and ring.

15. The finger prosthetic of claim 1, wherein the torsion spring is a 90-degree torsion spring and the hinge joint is passively extended by the torsion spring at a 90-degree angle, and the hinge joint is actively flexed via the cable.

16. A finger prosthetic comprising:
a fingertip portion and a midsection flexibly connected together through a hinge joint, the hinge joint further includes a plurality of inner knuckles and a plurality of outer knuckles connected by a pin therethrough;
a socket formed in the midsection for insertion of a user's limb;
a ring flexibly connected to the midsection by at least one lateral strut;
an embedded torsion spring embedded in the fingertip portion and midsection, the torsion spring configured to allow the fingertip portion to pivot relative to the midsection; and
a cable interwoven through the fingertip portion, the midsection and the ring; wherein when a user flexes a user's proximal interphalangeal (PIP) joint then tension is generated in the cable and the cable pulls on the fingertip portion flexing the midsection and the fingertip portion simultaneously to function as a prosthetic distal interphalangeal (DIP) joint.

17. The finger prosthetic of claim 16, wherein the torsion spring is a 90-degree torsion spring having a plurality of legs, and wherein the finger prosthetic uses flexion of a user's PIP joint at an interface with the midsection to create a tension in the cable.

18. The finger prosthetic of claim 17, wherein the tension compresses the legs of the torsion spring together and forces the device to bend, and when the user's PIP joint is relaxed the tension in the cable is released to cause the embedded torsion spring to return to a natural position at 90 degrees.

19. A method of using a finger prosthetic, comprising:
inserting a user's limb into a finger prosthetic having a ring, midsection and finger portion and a cable therethrough and a torsion spring equally embedded in both the finger portion and midsection, and wherein the midsection forms a socket;
positioning a distal end of the user's limb in the socket and positioning a base of the limb through the ring and thereround; and
generating tension on the cable by flexing a user's proximal interphalangeal (PIP) joint, wherein the cable pulls on the fingertip portion flexing the fingertip portion and the midsection simultaneously.

20. The method of claim 20, further comprising:
extending the user's PIP joint to causing the torsion spring to extend the fingertip portion and midsection; and
positioning the user's PIP joint at rest for the cable to release the tension in the torsion spring to cause the fingertip portion and midsection to extend to an upright position.
